(19) United States
(12) Patent Application Publication
(10) Pub. No.: US 2025/0205446 A1
(43) Pub. Date: Jun. 26, 2025

(54) HUMIDIFICATION VALUE COMPENSATION DEVICE, METHOD, AND RESPIRATORY DEVICE

(71) Applicant: RESVENT MEDICAL TECHNOLOGY CO., LTD, Shenzhen (CN)

(72) Inventors: Haiyang Pan, Shenzhen (CN); Rongrong Zhu, Shenzhen (CN)

(73) Assignee: RESVENT MEDICAL TECHNOLOGY CO., LTD, Shenzhen (CN)

(21) Appl. No.: 18/820,645

(22) Filed: Aug. 30, 2024

(30) Foreign Application Priority Data
Dec. 26, 2023 (CN) 202311815944X

(51) Int. Cl. A61M 16/16; A61M 16/00; A61M 16/10; G16H 40/63

(52) U.S. Cl. CPC A61M 16/161; A61M 16/024; A61M 16/109; G16H 40/63; A61M 2205/3334; A61M 2205/3368; A61M 2205/3393; A61M 2205/50

(57) ABSTRACT

A humidification value compensation device provided includes a sensor module and a main control module. The sensor module is configured to sense gas flow rate, environmental parameters, and the weight change value of the humidification tank during a predetermined humidification cycle. The environmental parameters include environmental temperature and humidity; The main control module is communicated with the sensor module, including a first calculation module, a second calculation module, an error calculation module, and a compensation value calculation module, which are configured to calculate an actual humidification value based on the gas volume, the weight change value, and the environmental parameters during the predetermined humidification cycle, calculate a target humidification value based on the environmental temperature and corresponding predetermined relative humidity, calculate a humidification error based on the actual humidification value and target humidification value, and calculate the humidification compensation value based on the humidification error and target humidification value.

This non-provisional patent application claims priority under 35 U.S.C. § 119 from Chinese Patent Application No. CN 202311815944.X filed on Dec. 26, 2023, the entire content of which is incorporated herein by reference.

### TECHNICAL FIELD

This application relates to the field of medical device technology, particularly to a respiratory device, and a humidification value compensation device and method thereof.

### BACKGROUND

Household ventilators have been widely used in respiratory support therapy, and humidifiers, as an important component of household ventilators, are mainly configured to appropriately humidify the transported gas and improve the problem of dry mouth and throat caused by long-term use of ventilators by users. Existing household ventilators often use heating devices to heat to a fixed temperature or dynamically adjust the temperature of the heating device based on environmental temperature and humidity to obtain the corresponding water temperature and complete humidification control. However, the mechanical connection between the heating device and the humidification tank cannot guarantee the consistency of their contact thermal conductivity. There is a deviation in the estimated water temperature based on the temperature of the heating device, resulting in a certain error between the humidification effect and the target value.

### SUMMARY

In view of this, it is necessary to provide a humidification value compensation device, method, and respiratory device for respiratory device.

Firstly, in accordance with an embodiment humidification value compensation device for a respiratory device is provided. The respiratory device comprises a heating pipeline and a humidification tank connected to the heating pipeline. The humidification value compensation device for the respiratory device comprises a sensor module and a main control module. The sensor module comprises a flow sensor, a temperature and humidity sensor, and a gravity sensor. The flow sensor is configured to sense the gas flow rate of the heating pipeline; The temperature and humidity sensor is configured to sense the environmental parameters of the respiratory device, including environmental temperature and humidity; The gravity sensor is configured to sense the weight change value of the humidification tank during a predetermined humidification cycle; The main control module is connected to the sensor module through communication, including a first calculation module, a second calculation module, an error calculation module, and a compensation value calculation module. The first calculation module is configured to calculate the gas volume during the predetermined humidification cycle based on the gas flow rate, and to calculate the actual humidification value based on the weight change value, gas volume, and environmental parameters; The second calculation module is configured to calculate the target humidification value based on the environmental temperature and corresponding predetermined relative humidity; The error calculation module is configured to calculate the humidification error based on the actual humidification value and the target humidification value; The compensation value calculation module is configured to calculate the humidification compensation value based on the humidification error and the target humidification value.

Secondly, in accordance with an embodiment a humidification value compensation method for the respiratory device applied to a humidification value compensation device for the respiratory device is provided. The respiratory device comprises a heating pipeline and a humidification tank connected to the heating pipeline. The humidification value compensation device for respiratory device comprises a sensor module and a main control module, the sensor module comprises a flow sensor, a temperature and humidity sensor, and a gravity sensor; The compensation method for the humidification value of the respiratory device includes: using the flow sensor, temperature and humidity sensor, and gravity sensor to sense the gas flow rate of the heating pipeline, the environmental parameters of the respiratory device, and the weight change value of the humidification tank during a predetermined humidification cycle, the environmental parameters include environmental temperature and humidity; calculating the gas volume during the predetermined humidification cycle based on the gas flow rate, and calculating the actual humidification value based on the weight change value, gas volume, and environmental parameters; calculating the target humidification value based on the environmental temperature and corresponding predetermined relative humidity; calculating the humidification error based on the actual humidification value and the target humidification value; and calculating the humidification compensation value based on the humidification error and the target humidification value.

Thirdly, in accordance with an embodiment a respiratory device is provided. The respiratory device includes a heating pipeline and a humidification tank connected to the heating pipeline. The respiratory device further comprises a sensor module and a main control module, wherein the sensor module comprises a flow sensor, a temperature and humidity sensor, and a gravity sensor; The main control module is communicated with the sensor module, including a memory and a processor, which are configured to store computer programs; The processor is configured to execute the computer program to implement the humidification value compensation method for the respiratory device mentioned above.

The humidification value compensation device, method, and respiratory device for the above-mentioned respiratory device, which detects the weight change value of the humidification tank that starts and ends humidification during the humidification cycle through a gravity sensor, and combines the gas flow rate of the heating pipeline sensed by the flow sensor and temperature and humidity sensor and the environmental parameters of the respiratory device to calculate the actual humidification value and target humidification value respectively during the humidification cycle to obtain the humidification error during the humidification cycle. Then, combined with the target humidification value, the humidification compensation value is obtained to complete the humidification compensation. The water consumption of the humidification tank during the humidification process is directly calculated through the gravity sensor, and the current humidification result is accurately calculated. Furthermore, the humidification error is further compensated for by calculating the humidification error. More accurate chemical control fundamentally solves the problem of inconsistent thermal conductivity between the heating device and the humidification tank, And reduced the mechanical contact requirements between the two.

### BRIEF DESCRIPTION OF THE DRAWINGS

In order to provide a clearer explanation of the embodiments or technical solutions in the disclosure or prior art, a brief introduction will be given below to the accompanying drawings required in the embodiments or prior art description. It is evident that the accompanying drawings in the following description are only some embodiments of the disclosure. For those skilled in the art, other accompanying drawings can be obtained based on the structures shown in these drawings without creative labor.

FIG. 1 is a schematic diagram of the structure of the humidification value compensation device of the respiratory device in accordance with an embodiment.

FIG. 2 is a schematic diagram of the respiratory device in accordance with an embodiment.

FIG. 3 is a schematic diagram of the humidification tank in accordance with an embodiment.

FIG. 4 is a schematic diagram of the heating device in accordance with an embodiment.

FIG. 5 is the first flowchart of the humidification value compensation method for the respiratory device in accordance with an embodiment.

FIG. 6 is a second flowchart of the humidification value compensation method for the respiratory device in accordance with an embodiment.

FIG. 7 is a schematic diagram of the internal structure of a respiratory device using a humidification value compensation method in accordance with an embodiment.

The implementation, functional characteristics, and advantages of the purpose of this application will be further explained in conjunction with the embodiments, with reference to the accompanying drawings.

### DETAILED DESCRIPTION OF THE EMBODIMENTS

In order to make the purpose, technical solution, and advantages of this application clearer and clearer, the following will provide further detailed explanations of this application in conjunction with the accompanying drawings and embodiments. It should be understood that the specific embodiments described here are only intended to explain the disclosure and are not intended to limit the disclosure. Based on the embodiments in this application, all other embodiments obtained by ordinary technical personnel in this field without creative labor fall within the scope of protection of this application.

The terms “first”, “second”, “third”, “fourth”, etc. (if any) in the specification and claims of this application, as well as the accompanying drawings, are configured to distinguish similar planning objects and do not need to be configured to describe specific order or sequence. It should be understood that the data used in this way can be interchanged in appropriate cases, in other words, the described embodiments are implemented in order other than those illustrated or described here. In addition, the terms “including” and “having”, as well as any variations thereof, may also include other content, such as processes, methods, systems, products, or equipment that include a series of steps or units, not necessarily limited to those clearly listed, but may include other steps or units that are not clearly listed or inherent to these processes, methods, products, or equipment.

It should be noted that the descriptions related to “first”, “second”, etc. in this application are only for descriptive purposes and cannot be understood as indicating or implying their relative importance or implying the quantity of technical features indicated. Therefore, the features limited to “first” and “second” can explicitly or implicitly include one or more of these features. In addition, the technical solutions between various embodiments can be combined with each other, but must be based on what ordinary technical personnel in the art can achieve. When the combination of technical solutions conflicts or cannot be achieved, it should be considered that the combination of such technical solutions does not exist and is not within the scope of protection required by this application.

Referring to FIG. 1, FIG. 1 illustrates a schematic diagram of a humidification value compensation device of a respiratory device provided in accordance with an embodiment. The humidification value compensation device 10 is, applied to a respiratory device 3 to measure the humidification compensation value of the respiratory device 3 during a humidification process. The humidification value compensation device 10 directly measures the weight changes of the humidification tank 32 at an beginning and an end of the humidification processes by a gravity sensor 13. Combined with relevant data sensed by other sensors, an actual humidification value and an target humidification value during the humidification process are calculated to determine the humidification compensation value, which is convenient to adjust the subsequent humidification process. The respiratory device 3 includes a heating pipeline 31, and a humidification tank 32 connected to the heating pipeline 31.

Referring to FIG. 1, the humidification value compensation device 10 includes a sensor module 1 and a main control module 2. The sensor module 1 includes a flow sensor 11 and a temperature and humidity sensor 12. The flow sensor 11 may be installed inside the heating pipeline 31 or at a junction between the heating pipeline 31 and the humidification tank 32 to sense the gas flow rate of the heating pipeline 31. The temperature and humidity sensor 12 is configured to sense the environmental parameters of respiratory device 3. Environmental parameters include environmental temperature and humidity.

In this embodiment, the sensor module 1 further includes a gravity sensor 13. The gravity sensor 13 is configured to sense a weight change value of the humidification tank 32 during a predetermined humidification cycle. The weight change value is a difference between a weight value of the humidification tank 32 at the beginning of the predetermined humidification cycle and a weight value of humidification tank 32 at the end of the predetermined humidification cycle. When the humidification tank 32 is heated at the beginning of the humidification process, the liquid inside the humidification tank 32 is heated and undergo a change in liquid weight. When humidification tank 32 is heated after a predetermined humidification cycle, the weight of the liquid in humidification tank 32 stops changing. An intuitive result of the humidification process is the change in the liquid inside the humidification tank 32, that is, the change in the weight of the humidification tank 32. Therefore, the measured weight change value of humidification tank 32 can intuitively reflect the change in liquid weight inside the humidification tank 32, that is, the liquid consumed by humidification tank 32 during the humidification process, making it easy to accurately determine the actual humidification value during the humidification process.

In this embodiment, the main control module may be a IC, or a firmware, etc. From functional blocks, the main control module 2 includes a first calculation module 21, a second calculation module 22, an error calculation module 23, and a compensation value calculation module 24. In this embodiment, the different calculation modules provided by the main control module 2 are configured to ultimately obtain the humidification compensation value for the humidification process. It will be understood that, the function of each module of the main control module is performed by a processor executing program instruction stored in a memory, the processor and the memory will be contained in one or more ICs or firmwares. A detail of each module in the main control module 2 will be described bellow.

In this embodiment, the first calculation module 21 is configured to calculate a gas volume during the predetermined humidification cycle based on the gas flow rate, and to calculate the actual humidification value based on the weight change value, the gas volume, and the environmental parameters. In detail, the calculation formula for gas volume is: V=flow*T, the V indicates gas volume, flow indicates gas flow rate, and T indicates the predetermined humidification period. The formula for calculating the actual humidification value is: Hₒᵤₜ=(m/V)+RH*(0.0387*temp²−0.6066*temp+13.776), the Hₒᵤₜ indicates the actual humidification value, the m indicates the weight change value, the RH indicates the environmental humidity, and temp indicates the environmental temperature.

In this embodiment, the second calculation module 22 is configured to calculate a target humidification value based on the environmental temperature and corresponding predetermined relative humidity. In detail, the calculation formula for the target humidification value is: aHₐᵢₘ=RHₐᵢₘ*(0.0387*temp²−0.6066*temp+13.776), the aHₐᵢₘ indicates the target humidification value, and the RHₐᵢₘ indicates the predetermined relative humidity corresponding to the environmental temperature.

In this embodiment, the error calculation module 23 is configured to calculate a humidification error based on the actual humidification value and the target humidification value. In detail, the calculation formula for humidification error is: error=Hₒᵤₜ−aHₐᵢₘ, the error indicates humidification error.

In this embodiment, the compensation value calculation module 24 is configured to calculate a humidification compensation value based on the humidification error and the target humidification value. In detail, the calculation formula for the humidification compensation value is: aH_{aim new}=aHₐᵢₘ+error, the aH_{aim new} indicates the humidification compensation value.

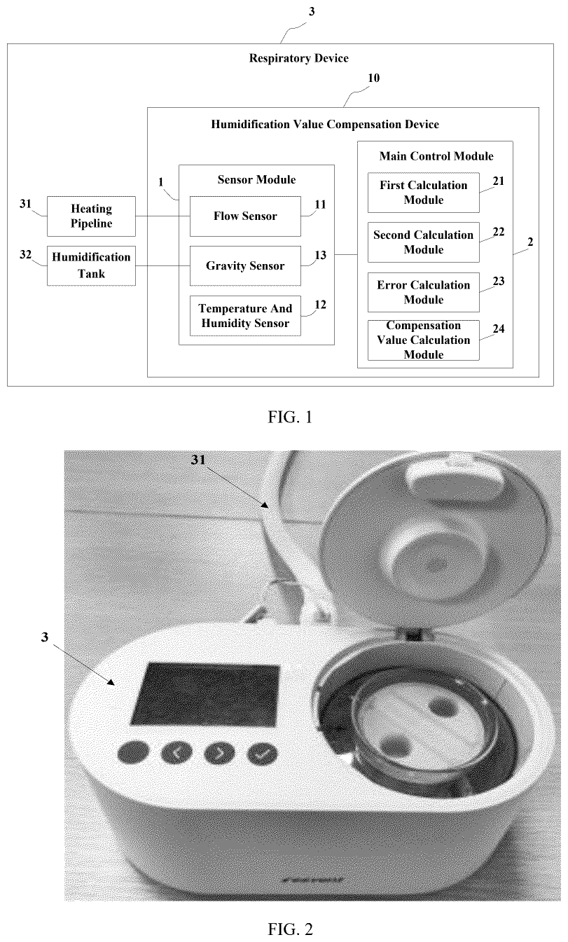

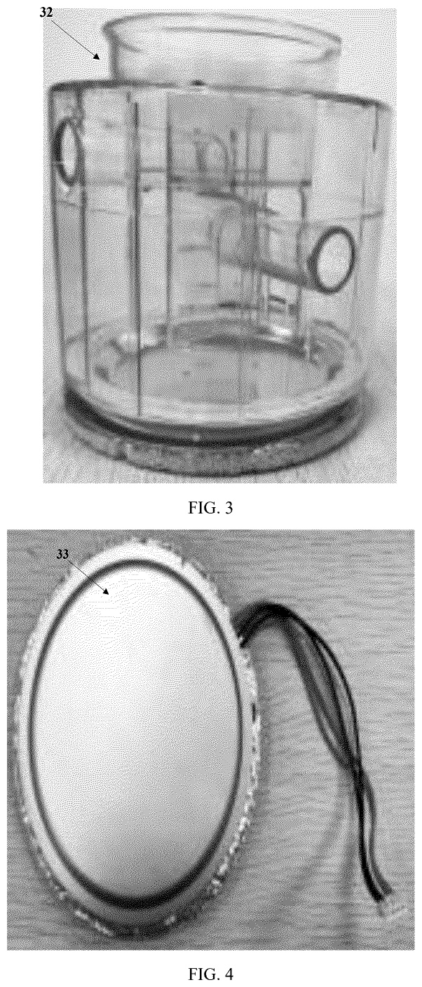

Referring to FIGS. 2-4. FIG. 2 illustrates a schematic diagram of the respiratory device provided in in accordance with an embodiment, FIG. 3 illustrates a schematic diagram of the humidification tank provided in in accordance with an embodiment, and FIG. 4 illustrates a schematic diagram of the heating device provided in in accordance with an embodiment.

The respiratory device 2 is configured to complete the humidification process through the provided humidification tank 32 and heating device 33 to measure the humidification value. In detail, a heating device 33 is disposed in the humidification tank 32. The heating device 33 includes but is not limited to a heating plate. The heating device 33 is mechanically connected to a bottom of the humidification tank 32 according to a predetermined heating method to heat the humidification tank 32 during the predetermined humidification cycle. Due to the intuitive manifestation of the humidification process as the weight change of the humidification tank 32, and the connection between the heating device 33 and the humidification tank 32, in order to accurately sense the weight change of the humidification tank 32 during the humidification process and prevent the gravity sensor 13 from being excessively affected by heat due to being directly installed at the bottom of the humidification tank 32, the gravity sensor 13 can be mounted at the bottom of the heating device 33.

In this embodiment, the compensation value calculation module 24 is also configured to obtain a target temperature of the heating device 33 based on the humidification compensation value and a predetermined relationship, and to adjust the current heating temperature of the heating device 33 to the target temperature. The predetermined relationship is a relationship between the environmental temperature, the target humidification value, and the target temperature. That is, in order for respiratory device 3 to have the target humidification value during the humidification process at a certain environmental temperature to achieve the expected humidification effect, the current heating temperature of heating device 33 needs to be adjusted to the target temperature. The predetermined relative humidity is determined by the environmental temperature based on the predetermined corresponding relationship.

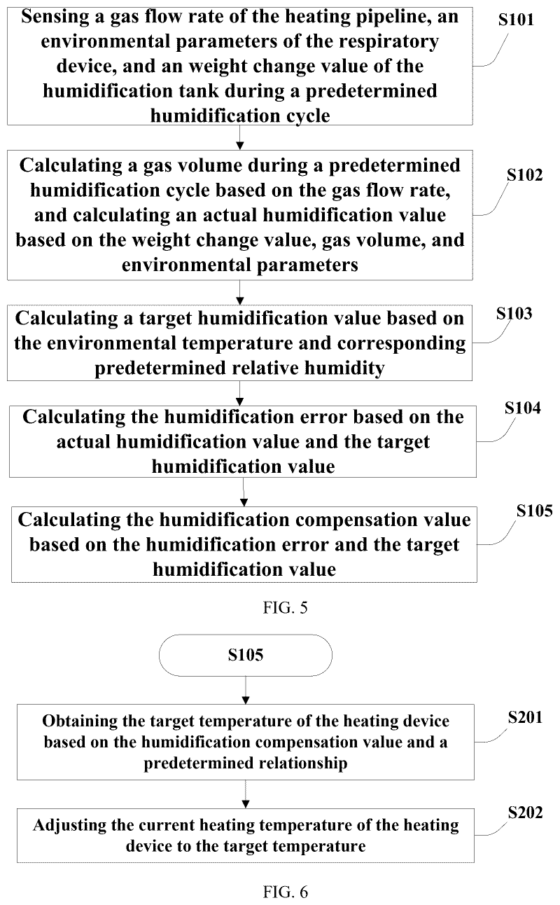

Referring to FIG. 5, FIG. 5 illustrate a first flowchart of the humidification value compensation method for the respiratory device in accordance with an embodiment. This application also provides a humidification value compensation method for respiratory device, applied to a humidification value compensation device 10 for respiratory device. The heating pipeline 31 and humidification tank 32 of respiratory device 3 have been explained above and will not be elaborated. The humidification value compensation device 10 includes a sensor module 1 and a main control module 2. The sensor module 1 includes a flow sensor 11, a temperature and humidity sensor 12, and a gravity sensor 13. The specific characteristics of each component in the humidification value compensation device 10 have been described in detail above and will not be elaborated here. The humidification value compensation method for respiratory device includes steps S101-S105.

In the step S101, by a flow sensors, a temperature and humidity sensors, and a gravity sensors, a gas flow rate in the heating pipeline, environmental parameters of the breathing equipment, and the weight change value of the humidification tank during the predetermined humidification cycle are sensed.

In detail, in the step S101, the environmental parameters include environmental temperature and environmental humidity. The weight change value is the difference between the weight value of humidification tank 32 at the beginning of the predetermined humidification cycle and the weight value of humidification tank 32 at the end of the predetermined humidification cycle.

In the step S102, a gas volume during the predetermined humidification cycle is calculated based on the gas flow rate, and calculate the actual humidification value based on the weight change value, gas volume, and environmental parameters.

In detail, in the step S102, the actual humidification value can be calculated using a predetermined formula. In detail, the predetermined formula for calculating the gas volume is: V=flow*T, the V indicates gas volume, the flow indicates gas flow rate, and the T indicates the predetermined humidification period. A predetermined formula for calculating the actual humidification value is: Hₒᵤₜ=(m/V)+RH*(0.0387*temp²−−0.6066*temp+13.776), the Hₒᵤₜ indicates the actual humidification value, the m indicates the weight change value, the RH indicates the environmental humidity, and the temp indicates the environmental temperature.

In the step S103, a target humidification value is calculated based on the environmental temperature and corresponding predetermined relative humidity.

In detail, in the step S103, the target humidification value can be calculated using a predetermined formula. In detail, the predetermined formula for calculating the target humidification value is: aHₐᵢₘ=RHₐᵢₘ*(0.0387*temp²−0.6066*temp+13.776), the aHₐᵢₘ indicates the target humidification value, and the RHₐᵢₘ indicates the predetermined relative humidity corresponding to the environmental temperature.

In step S104, the humidification error is calculated based on the actual humidification value and the target humidification value.

In detail, in the step S104, a humidification error can be calculated using predetermined formula. In detail, the predetermined formula for calculating the humidification error is: error=Hₒᵤₜ−aHₐᵢₘ, the error indicates humidification error.

In step S105, a humidification compensation value is calculated based on the humidification error and target humidification value.

In detail, in the step S105, the humidification compensation value can be calculated using a predetermined formula. In detail, the predetermined formula for calculating the humidification compensation value is: aH_{aim new}=aHₐᵢₘ+error, the aH_{aim new} indicates the humidification compensation value.

In this embodiment, the humidification tank 32 is equipped with a heating device 33. The heating device 33 is mechanically connected to the bottom of the humidification tank 32 according to the predetermined heating method to heat the humidification tank 32 during the predetermined humidification cycle. Due to the intuitive manifestation of the humidification process as the weight change of the humidification tank 32, and the connection between the heating device 33 and the humidification tank 32, in order to accurately sense the weight change of the humidification tank 32 during the humidification process and prevent the gravity sensor 13 from being excessively heated due to being directly located at the bottom of the humidification tank 32, the gravity sensor 13 can be set at the bottom of the heating device 33 to accurately sense the weight change of the humidification tank 32 at the beginning and end of the predetermined humidification cycle, and obtain the weight change value.

Referring to FIG. 6, FIG. 6 illustrates a second flowchart of the humidification value compensation method for the respiratory device provided in in accordance with an embodiment. The humidification value compensation method for respiratory device also includes steps S201-S202.

In step S201, a target temperature of the heating device is obtained based on the humidification compensation value and the predetermined corresponding relationship.

In detail, in the step S201, a predetermined relationship is the relationship between environmental temperature, target humidification value, and target temperature.

In detail, in the step S201, the predetermined relationship is the correspondence between environmental temperature, target humidification value, and target temperature. That is, in order for respiratory device 3 to have the target humidification value during the humidification process at a certain environmental temperature to achieve the expected humidification effect, the current heating temperature of heating device 33 needs to be adjusted to the target temperature. The predetermined relative humidity is determined by the environmental temperature based on the predetermined corresponding relationship.

S202, the current heating temperature of the heating device is adjusted to the target temperature.

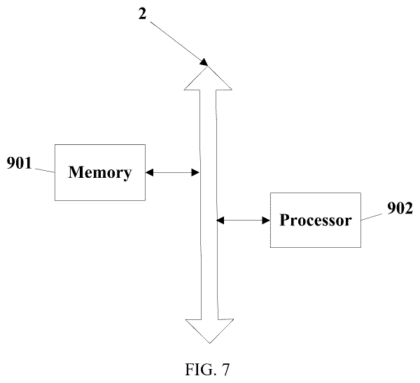

Referring to FIG. 7, FIG. 7 illustrates a schematic diagram of the internal structure of the respiratory device provided in the embodiment of the disclosure, using the humidification value compensation method of the respiratory device.

As shown in FIG. 7, the disclosure also provides a respiratory device 3. The respiratory device 3 includes a heating pipeline 31, and a humidification tank 32 connected to the heating pipeline 31. The Respiratory device 3 also includes sensor module 1 and main control module 2. The sensor module 1 includes a flow sensor 11, a temperature and humidity sensor 12, and a gravity sensor 13. The main control module 2 is communicated with the sensor module 1, which means that respiratory device 3 includes at least the humidification value compensation device 10 of the aforementioned respiratory device. The specific characteristics of each component in the humidification value compensation device 10 have been elaborated in detail in the previous text and will not be elaborated here. The main control module 2 also includes a memory 901 and a processor 902. The processor 902 is configured to run computer program instructions in memory 901 to perform a humidification value compensation method for respiratory device.

The memory 901 includes at least one type of readable storage medium, which includes flash memory, hard disk, multimedia card, card type memory (such as SD or DX memory, etc.), magnetic memory, magnetic disk, optical disc, etc. In some embodiments, memory 901 may be an internal storage unit of a computer device, such as a hard disk of the computer device. In other embodiments, the memory 901 can also be a storage device of an external computer device, such as a plug-in hard disk configured on the computer device, a Smart Media Card (SMC), a Secure Digital (SD) card, a Flash Card, etc. Furthermore, the memory 901 may include both internal storage units of computer devices and external storage devices. Memory 901 can not only be configured to store application software installed on computer devices and various types of data, such as codes for humidity compensation methods of respiratory devices, but can also be configured to temporarily store data that has already been or will be output.

FIG. 7 only illustrates the respiratory device 3 with partial components and a method for compensating the humidification value of the respiratory device. Those skilled in the art can understand that the structure shown in FIG. 7 does not constitute a limitation on the respiratory device 3, and may include fewer or more components than shown, or combinations of certain components, or different component arrangements.

In the above implementation example, the weight value of the humidification tank that starts and ends humidification during the humidification cycle is sensed by a gravity sensor to obtain the weight change value. Combined with the gas flow rate of the heating pipeline sensed by the flow sensor and the temperature and humidity sensor, and the environmental parameters of the breathing equipment, the actual humidification value and target humidification value during the humidification cycle are separately calculated to obtain the humidification error during the humidification cycle. Then, combined with the target humidification value, the humidification compensation value is obtained to complete the humidification compensation. The water consumption of the humidification tank during the humidification process is directly calculated by the gravity sensor, and the current humidification result is accurately calculated. Furthermore, the humidification error is further compensated for, making humidification control more accurate and fundamentally improving the humidification control. Solved the problem of inconsistent thermal conductivity between the heating device and the humidification tank, and reduced the mechanical contact requirements between the two.

Obviously, technical personnel in this field can make various modifications and variations to the disclosure without departing from the spirit and scope of the disclosure. In this way, if these modifications and variations of the disclosure fall within the scope of the claims and their equivalent technologies, the disclosure also intends to include these modifications and variations.

It should be understood that although the various steps in the flowchart in the attached figure are displayed sequentially according to the arrows, these steps are not necessarily executed in the order indicated by the arrows. Unless explicitly stated in this article, the execution of these steps does not have strict order restrictions and can be carried out in other orders. Moreover, at least a portion of the steps in the flowchart of the attached figure may include multiple sub steps or stages, which may not necessarily be completed at the same time, but may be executed at different times, and their execution order may not necessarily be sequential, but may rotate or alternate with at least a portion of other steps or sub steps or stages.

The above listed examples are only the preferred embodiments of this application, and of course, they cannot be configured to limit the scope of the rights of this application. Therefore, the equivalent changes made according to the claims of this application still fall within the scope of this application.

1. A humidification value compensation device for a respiratory device, comprising a heating pipeline, and a humidification tank and connected to the heating pipeline, wherein a heating element is disposed on the humidification tank to heating the humidification tank during a predetermined humidification cycle, the humidification value compensation device for the respiratory device comprises:
a sensor module comprising:
a temperature and humidity sensor, configured to sense environmental parameters of the respiratory device, the environmental parameters including environmental temperature and environmental humidity, and
a gravity sensor, disposed on the heating element configured to sense a weight change value of the humidification tank during the predetermined humidification cycle, the weight change value is a difference between the weight value of the humidification tank at beginning of the predetermined humidification cycle and the weight value of the humidification tank when at an end of the predetermined humidification cycle;
a flow sensor, configured to sense a gas flow rate of the heating pipeline;
a memory, configured to store program instruction; and
a main control module, communicated with the sensor module, and configured to executing the program instructions to perform a humidification value compensation method comprising:
calculating a gas volume during a predetermined humidification cycle based on the gas flow rate, and calculating an actual humidification value based on the weight change value, gas volume, and environmental parameters;
calculating a target humidification value based on the environmental temperature and a corresponding predetermined relative humidity;
calculating a humidification error based on the actual humidification value and the target humidification value; and
calculating a humidification compensation value based on the humidification error and the target humidification value.

2. The humidification value compensation device for the respiratory device of claim 1, wherein the humidification value compensation further comprises:
obtaining the target temperature of the heating device based on the humidification compensation value and a predetermined relationship, and
adjusting the current heating temperature of the heating device to the target temperature, the predetermined relationship is a relationship between the environmental temperature, the target humidification value, and the target temperature.

3. The humidification value compensation device for the respiratory device of claim 1, wherein the heating element is mounted on a bottom of the humidification tank.

4. The humidification value compensation device for the respiratory device of claim 3, wherein the gravity sensor is mounted on a bottom of the heating element.

5. The humidification value compensation device for the respiratory device of claim 1, wherein a calculation formula for the target humidification value is: aHₐᵢₘ=RHₐᵢₘ*(0.0387*temp²−0.6066*temp−13.776), the aHₐᵢₘ indicates the target humidification value, the temp indicates the environmental temperature, and the RHₐᵢₘ indicates the predetermined relative humidity corresponding to the environmental temperature; a calculation formula for the humidification error is: error=Hₒᵤₜ−aHₐᵢₘ, the error indicates the humidification error, the Hₒᵤₜ indicates the actual humidification value; a calculation formula for the humidification compensation value is: aH_{aim new}=aHₐᵢₘ+error, the aH_{aim new} indicates the humidification compensation value.

6. A humidification value compensation method for a respiratory device, the respiratory device comprising a heating pipeline and a humidification tank connected to the heating pipeline, the humidification value compensation method of the respiratory device comprising:
sensing a gas flow rate of the heating pipeline, an environmental parameters of the respiratory device, and an weight change value of the humidification tank during a predetermined humidification cycle, the environmental parameters include environmental temperature and humidity, the weight change value is a difference between the weight value of the humidification tank at beginning of the predetermined humidification cycle and the weight value of the humidification tank when at an end of the predetermined humidification cycle;
calculating a gas volume during the predetermined humidification cycle based on the gas flow rate, and calculating an actual humidification value based on the weight change value, gas volume, and environmental parameters;
calculating a target humidification value based on the environmental temperature and corresponding predetermined relative humidity;
calculating the humidification error based on the actual humidification value and the target humidification value; and
calculating the humidification compensation value based on the humidification error and the target humidification value.

7. The humidification value compensation method for the respiratory device of claim 6, further comprising:
obtaining the target temperature of the heating device based on the humidification compensation value and a predetermined relationship, the predetermined relationship is a relationship between the environmental temperature, the target humidification value, and the target temperature; and
adjusting the current heating temperature of the heating device to the target temperature.

8. The humidification value compensation method for the respiratory device of claim 6, wherein a calculation formula for the gas volume is: V=flow*T, the V indicates the gas volume, the flow indicates the gas flow rate, and the T indicates the predetermined humidification period; a calculation formula for the actual humidification value is: Hₒᵤₜ=(m/V)+RH*(0.0387*temp²−0.6066*temp+13.776), wherein the Hₒᵤₜ indicates the actual humidification value, the m indicates the weight change value, the RH indicates the environmental humidity, and temp indicates the environmental temperature.

9. The humidification value compensation method for a respiratory device of claim 8, wherein a calculation formula for the target humidification value is: aHₐᵢₘ=RHₐᵢₘ*(0.0387*temp²−0.6066*temp−13.776), the aHₐᵢₘ indicates the target humidification value, the temp indicates the environmental temperature, and the RHₐᵢₘ indicates the predetermined relative humidity corresponding to the environmental temperature; a calculation formula for the humidification error is: error=Hₒᵤₜ−aHₐᵢₘ, the error indicates the humidification error; a calculation formula for the humidification compensation value is: aH_{aim new}−aHₐᵢₘ+error, the aH_{aim new} indicates the humidification compensation value.

10. A respiratory device comprising a heating pipeline and a humidification tank connected to the heating pipeline, the respiratory device further comprising:
a sensor module comprising:
a temperature and humidity sensor, configured to sense environmental parameters of the respiratory device, the environmental parameters including environmental temperature and environmental humidity, and
a gravity sensor, disposed on the heating element configured to sense a weight change value of the humidification tank during the predetermined humidification cycle, the weight change value is a difference between the weight value of the humidification tank at beginning of the predetermined humidification cycle and the weight value of the humidification tank when at an end of the predetermined humidification cycle;
a flow sensor, configured to sense a gas flow rate of the heating pipeline;
a memory, configured to store program instruction; and
a processor, configured to executing the program instructions to perform a humidification value compensation method comprising:
calculating a gas volume during a predetermined humidification cycle based on the gas flow rate, and calculating an actual humidification value based on the weight change value, gas volume, and environmental parameters;
calculating a target humidification value based on the environmental temperature and a corresponding predetermined relative humidity;
calculating a humidification error based on the actual humidification value and the target humidification value; and
calculating a humidification compensation value based on the humidification error and the target humidification value.

11. The humidification value compensation device for the respiratory device of claim 10, wherein the humidification value compensation further comprises:
obtaining the target temperature of the heating device based on the humidification compensation value and a predetermined relationship; and
adjusting the current heating temperature of the heating device to the target temperature, the predetermined relationship is a relationship between the environmental temperature, the target humidification value, and the target temperature.

12. The respiratory device of claim 10, wherein the heating element is mounted on a bottom of the humidification tank.

13. The respiratory device of claim 12, wherein the gravity sensor is mounted on a bottom of the heating element.

14. The respiratory device of claim 10, wherein a calculation formula for a target humidification value is: aHₐᵢₘ=RHₐᵢₘ*(0.0387*temp²−0.6066*temp−13.776), the aHₐᵢₘ indicates the target humidification value, the temp indicates the environmental temperature, and the RHₐᵢₘ indicates the predetermined relative humidity corresponding to the environmental temperature; a calculation formula for the humidification error is: error=Hₒᵤₜ−aHₐᵢₘ, the error indicates the humidification error, the Hₒᵤₜ indicates the actual humidification value; a calculation formula for the humidification compensation value is: aH_{aim new}=aHₐᵢₘ+error, the aH_{aim new} indicates the humidification compensation value.
